(19) United States
(12) Patent Application Publication
(10) Pub. No.: US 2003/0046733 A1
(43) Pub. Date: Mar. 6, 2003

(54) Transformation of soybeans

(76) Inventor: Kalyani Mallika Dias, Ames, IA (US)

Correspondence Address: MARK A. LITMAN & ASSOCIATES, P.A., York Business Center, Suite 205, 3209 W. 76th ST., Edina, MN, 55402, US

(21) Appl. No.: 09/948,292

(22) Filed: Sep. 6, 2001

(51) Int. Cl. C12N 15/82; C12N 15/87; A01H 5/00; C12N 15/84

(52) U.S. Cl. 800/294; 800/312; 800/292; 800/293; 435/469; 435/470

(57) ABSTRACT

Soybean are transformed by inserting a functional gene into an explant of a soybean (particularly after being pre-treated with high doses of cytokinin (hormone)), transferring embryonic axes explants of the mature soybean seeds incubated on wet filter papers in the presence of at least one phenol compound naturally produced when plant cells have been wounded, to induce vir genes, and incubated in the dark in such presence at 20° C.-25° C. for at least 24 hr. After incubation, the explants are transferred to a media to develop shoots from explants, control Agrobaterium growth, and after shoot elongation, separated shoots, with or without roots, are either transferred to soil, or contacted with at least 1 mg/l IBA before transplant.

### BACKGROUND OF THE INVENTION

1. Field of the Invention

This invention pertains to a method for regenerating plants from transformed soybeans. Specifically, a method of regenerating soybeans, using transformed embryonic axes explants to effect genetic transformation of a large variety of cultivars, coupled with plant regeneration from the embryonic axes, provides a reliable means for introducing genetic variation.

2. Background of the Art

Soybean [Glycine max (L.) Merr.] is one of the world's most important agronomic crops. Between 120 and 130 million acres are planted annually, resulting in 105 million tons of seed. Soybeans have dominated world oilseed production among the eight major oilseeds traded in international markets, accounting for over 97% of all world oilseed production since 1965. The value of the crop is estimated to be over 20 billion dollars. Both soybean oil and protein are used extensively in food products for human consumption. In the United States, 5% of the total protein is derived from grain legumes and up to 65% of the oil used by the food processing industry comes from soybean (Hoskin, USDA Econ. Res. 1-35 (1987), Smith and Huyser, 1-22 (1987).

A great deal of effort has been made towards the development of new cultivars of soybean with improved disease, pest, herbicide resistance along with increased nutritional value. However traditional breeding programs have been restricted because soybean germplasm is extremely narrow and the majority of the soybean cultivars in use are derived from very few parental lines Christou et al., TIBTECH 8:145-151 (1990).

Also only a few plant introductions have given rise to the major cultivars grown in the United States and, as a consequence, this narrow germplasm base has limited soybean breeding. Hence, modification of soybean using genetic engineering techniques would facilitate the development of new varieties with traits such as herbicide resistance, disease resistance such as virus resistance and seed quality improvement in a manner unattainable by traditional breeding methods or tissue-culture induced variation. Genes have been transferred to soybean protoplasts by electroporation of free DNA (Christou et al., 1987 and Lin et al., 1987). However, regeneration technology for soybean has not progressed to a state where regenerated plants can be produced from protoplasts. Soybean shoot organogenesis occurs from tissues such as cotyledonary nodes (Cheng et al., 1980; Wright et al., 1986; and Barwale et al., 1986) as well as primary leaves of seedlings (Wright et al., 1987). Somatic embryogenesis has been demonstrated from immature embryos and cotyledons of developing seeds (Ranch et al., 1985; Lazzeri et al., 1985; Ghazi et al., 1986; Barwale et al., 1986; and Hammat et al., 1987).

The development of gene transformation techniques has created an alternate path to the genetic improvement of many crops such as increased disease, pest and herbicide resistance, as well as quality improvement. However, success with these approaches has been limited in soybean due to various in vitro difficulties attributed to soybean Christou, supra. The design of genetic engineering protocols for soybean would include the development of an efficient plant regeneration system.

The majority of reports on soybean regeneration utilized cotyledons from immature zygotic embryos induced to undergo somatic embryogenesis Liu et al., In Vitro Cell. Dev. Biol. 28P: 153-160 (1992), which could entail protracted culture periods. Soybean regeneration through short meristem cultures resulted in up to 35% explants responding to plant regeneration 4 weeks after culture Kartha et al., Can. J. Bot., 59:1671-1679 (1981). Regeneration via organogenesis utilizing different explants has been reported with a maximum of 97% of explants responding and 3 shoots produced per explant 10 weeks after culture, and 38% of shoots developing roots for another 4 weeks Yeh et al., J. Argric. Assoc. China, 40:77-90 (1991). However, interactions between genotype and in vitro cultural conditions (medium, explant and light treatment) have not been reported in regeneration via organogenesis or meristem culture in soybean, although it has been studied in regeneration via somatic embryogenesis and was proven important Powell et al., Heredity 58:75-801 (1987); Komatsuda et al., Crop. Sci. 31:333-337 (1991).

U.S. Pat. No. 5,322,783, Tomes et al., is directed to a method for transformation of soybean tissue which calls for treating cotyledonary node cells with a cytokinin, and then bombarding the cells with microparticles carrying specific vectors and exgenogeous DNA. U.S. Pat. Nos. 5,169,770 and 5,376,543, Chee et al., focus on a different method of transforming soybeans, to produce transgenic plants. In the process described in these patents, seeds are germinated, and the meristematic or mesocotyl cell tissues are inoculated with bacterial cells, specifically Agrobacterium strains, which through infection, transfers DNA into these explants. Transgenic plants could ensue provided this transfection transformation was successful and occured prior to differentiation.

Any plant transformation program also requires a regeneration program. In U.S. Pat. No. 4,992,375, Wright et al., a process is described which calls for excising the cotyledonary node region from a donor plant, and culturing the explant in a nutrient media containing cytokinin, until shoots arose from resultant callus. The shoots are then rooted. U.S. Pat. No. 5,416,011, Hinchee et al., also utilizes a cotyledon explant, which requires removal of the hypocotyl, saving and separating the cotyledons and inserting a chimeric gene by inoculation with Agrobacterium tumefaciens vectors containing the desired gene. This reference, and many others, employ the GUS histochemical marker to determine successful transformation.

Generally, the processes developed are categorized by a variety of inadequacies. Reliable transformation and regeneration is not accomplished. The formation of shoots, and eventual rooting, takes place only in a tiny fraction of the cases. Further, successful transformation and successful regeneration are frequently cultivar-specific, with no broad success. Among investigators reporting random, and overall poor success, are Wayne et al., Plant Mol. Biol. (1988); Finer et al., In Vitro Cell. Dev. Biol. (1991); Sato et al., Plant Cell Reports (1993); Moore et al., Plant Cells Reports (1994); Parrott et al., In Vitro Cell. Dev. Biol. (1994) and Stewart et al., Plant Physiol. (1996). While limited successes in producing transgenic plants are reported (e.g., 1 out of 195 in Parrott et al. 1994), success is random, and not predictable.

U.S. Pat. No. 5,824,877 describes a method for transforming soybean which comprises: (a) preparing a cotyledon explant from a soybean seedling by:

(i) removing the hypocotyl region by cutting just below the cotyledonary node,

(ii) separating the two cotyledons at the cotyledonary node by tearing the cotyledons apart, and

(iii) removing the epicotyl from the cotyledon to which it remains attached,

(b) inserting a chimeric gene into the explant of part (a) by inoculation and co-cultivation of the explant with a disarmed Agrobacterium tumefaciens vector containing said chimeric gene;

(c) selecting transformed explant tissue, and

(d) regenerating a differentiated transformed plant from the transformed explant tissue of part (c).

U.S. Pat. No. 5,569,834 describes a method for producing a soybean plant comprising a chimeric gene and associated DNA resulting from an Agrobacterium tumefaciens-mediated transformation, said chimeric gene capable of conferring kanamycin resistance to said soybean plant, the method comprising:

(a) preparing a cotyledon explant from a soybean seedling by:

(i) removing the hypocotyl region by cutting just below the cotyledonary node,

(ii) separating the two cotyledons at the cotyledonary node, and

(iii) removing the epicotyl from the cotyledon to which it remains attached,

(b) inserting a chimeric gene into the explant of part (a) by inoculation and co-cultivation of the explant with a disarmed Agrobacterium tumefaciens vector containing said chimeric gene;

(c) selecting transformed explant tissue, and

(d) regenerating a differentiated transformed plant from the transformed explant tissue of part (c).

U.S. Pat. No. 5,968,830 describes a method for transformation and regeneration of soybeans. The process comprises regenerating soybeans (G. max) via organogenesis, comprising: obtaining hypocotyls explants from germinated seedlings of soybean plants whose regeneration is desired, maintaining said hypocotyls explants on a shoot induction medium comprising a cytokinin until shoots form at the acropetal end of said hypocotyls explant, excising shoots from said hypocotyls explant and maintaining said shoots on a shoot elongation medium until said shoots are competent on a rooting medium and maintaining said shoots on a rooting medium until rooted plantlets are formed, and transplanting said plantlets to soil. Also describes is a method for transforming soybean plants to express exogenous DNA, comprising: obtaining a hypocotyls explant from germinated seedlings of a soybean plant, maintaining said explant on a shoot induction medium comprising a cytokinin for 16-32 hours, bombarding said explant, with the acropetal end facing up, with microparticles of an inert metal coated with exogenous DNA comprising a plasmid which comprises an expression gene, said expression gene encoding the expression of a protein exogenous to said soybean plant, maintaining said bombarded Hypocotyls explant on a medium selective for growth of transformed tissues, followed by maintenance on a shoot elongation medium and preparing plantlets from shoots so obtained.

Accordingly, it remains an object of those of skill in the art to develop a reliable, repeatable and non-cultivar-limited method for transforming and regenerating soybeans.

### SUMMARY OF THE INVENTION

The present invention is a simple, rapid, and reliable process for producing transgenic soybean plants. The method is effective with all soybean varieties, including elite commercial cultivars, and shows a substantial improvement in the regeneration from the transgenic tissues over previously-used systems because it provides necessary and hereto-missing factors for transformation and regeneration of soybeans, and optimizes these and other factors for the successful production of healthy, fertile transgenic plants. This invention may comprise a genotype-independent method for producing transgenic soybean plants comprising:

(a) Pre-treating sterile seeds with a relatively high concentration (e.g., at least about 40, preferably at least 35 mg/L, and 50-100 mg/L) of BAP (6-benzylaminopurine) for at least 1.5 hours, preferably at least 2 or 2.5 hours, most preferably about 3 hr;

(b) co-cultivating an explant derived from embryonic axes of a mature soybean seed with Agrobacterium species containing a chimeric gene in the presence of liquid plant tissue culture media;

(C) inducing virulence of the Agrobacterium by incubating the tissues at a temperature between 22° C.-24° C.;

(d) incubating co-cultivated explants on a support medium such as filter-paper to increase dependency of the bacteria on plant cells as well as to reduce the excessive growth of Agrobacterium that normally occurs on agar medium;

(e) inducing virulence of the Agrobacterium by soaking the support medium (e.g., the filter-paper) with a signal compound selected from the group consisting of acetosyringone, alpha.-hydroxyacetosyringone, acetovanillone. syringaldehyde, syringic acid, and sinapinic acid and mixtures thereof; and

(f) growing the co-cultivated tissue in growth regulator free media with a selection agent to induce shoots.

This process involves preferably soaking pre-sterilized seeds in about 50 to 100 mg/l BAP (6-benzylaminopurine) containing MS liquid media for about 3 hr. and dissecting the seeds and excising the embryonic axes explants for co-cultivation. A soybean explant is a piece of soybean tissue that is taken from a donor plant and is capable of producing adventitious shoots in culture. Soybean embryonic axes tissue is that portion of the embryo of a seed when the apical meristematic region (growth point including the first leaves) is removed

The process also may be described as including co-cultivation of the soybean embryonic axes explants with an Agrobacterium species (culture concentration is 1.5×10⁸ cells/mL) carrying a plasmid into which is inserted the gene or genes of interest for about 30 min. Co-cultivation is a process where the plant tissues are soaked in Agrobacterium. After the 30 minute soaking, plant tissues are removed from the bacteria culture and placed on wet filter paper, and soaked with acetosyringone, a phenol compound produced naturally and secreted by wounded plant cells, to induce the vir genes.

The process may also be described as a method for transforming and regenerating soybean comprising:

a) inserting a functional gene into a cell of a soybean explant of a soybean seed;

b) explants of the soybean seeds developed from step b) are then transferred to a porous substrate for incubation;

c) after incubation, the explants are transferred to a solution of macro and micro salts supplemented with 1×B5 vitamins and a selection agent; and

d) after shoot elongation, separated shoots, with or without roots, are either

i. transferred to soil; or

ii. contacted with at least 1 mg/l IBA before transplant.

Agrobacterium may be used for inserting the functional gene into the cell. In that event, embryonic axes explants of the soybean seeds are developed in step a) are co-cultivated and are transferred to the porous substrate. Also the transfer to the porous substrate is carried out in the presence of at least one phenol compound naturally produced when plant cells have been wounded, to induce vir genes. The porous medium may also contain a signal compound. Non-limiting examples of the signal compound are selected from the group consisting of acetosyringone, alpha-hydroxyacetosyringone, acetovillone, syringaldehyde, syringic acid, sinapinic acid, and mixtures thereof. Incubation is preferably performed in the dark in the presence of the at least one phenol compound at 20° C.-25° C. for at least 24 hours. After the incubation shoot development may be performed in the presence of an antibiotic to control Agrobacterium growth. Also, the shoot development may be performed in the presence of a selection agent. Non-limiting examples of the selection agent comprises an antibiotic or an herbicide. Non-limiting examples of these include kanamycin, bialophos, hygromycin, and glyphosate.

Another way of describing the process is a method of inducing shoots from transgenic soybean tissues by pretreating seeds that leads to development into a transgenic soybean plant comprising:

e) pre-treating sterile seeds with a relatively high concentration of at least about 40 mg/L of 6-benzylaminopurine for at least 1.5 hours.

A non-limiting example of a relatively high concentration of a solution of 6-benzylaminopurine between about 40-100 mg/L. A preferred pretreatment is performed for at least 2.5 hours. After the pretreatment, it may be preferred if an explant derived from embryonic axes of a mature soybean seed is co-cultivated with Agrobacterium species containing a chimeric gene in the presence of liquid plant tissue culture media. Also, an option is for after the pretreatment, an explant derived from embryonic axes of a mature soybean seed is co-cultivated with Agrobacterium species containing a chimeric gene in the presence of liquid plant tissue culture media. For example, virulence may be induced in the Agrobacterium by incubating the tissues at a temperature between 20° C.-25° C. It is also possible for co-cultivation to be performed on porous support media. The process may have porous support medium contain a signal compound, such as those non-limiting examples selected from the group consisting of acetosyringone, alpha-hydroxyacetosyringone, acetovanillone. syringaldehyde, syringic acid, sinapinic acid, and mixtures thereof.

Incubating the co-cultivated tissues on the porous support medium (e.g., the filter-paper) has improved the recovery of the transgenic plants. It is observed that when there is no plant or bacteria media for the Agrobacterium to depend on, the infection efficiency increased. At the same time, the lower growth of bacteria helps in reducing the overgrowth of remaining bacteria in the plant media once the tissues are transferred to the media after incubation.

Previous studies have recognized the importance of vir gene induction for soybean transformation but failed to solve the problems encountered in effecting that process. Delzer, et al. (1990) Crop Sci. 30. 320-322: Owens and Smigocki (1988) Plant Physiol. 88. 570-573. This invention uses acetosyringone, a phenol compound produced by wounded plant cells, to induce the vir genes. Other phenolic compounds, alpha.-hydroxyacetosyringone, acetovanillone, syringaldehyde, syringic acid, and sinapinic acid, also induce the vir genes and their use individually or in combination with acetosyringone can result in improved soybean transformation efficiencies.

The plant tissues on the wet filter paper are incubated in the dark, e.g., 20° C. to 25° C. or at 22° C. to 24° C. for two days.

The temperature for co-cultivation is known to be another important factor. The usual temperatures (26° C. to 28° C.) for the Agrobacterium culture is not suitable for soybean cells. Therefore co-cultivating at a lower temperature (20° C. to 25° C., especially at 22° C. to 24° C.) resulted in effective transformation.

After transformation, explants are cultivated in culture medium contains counter-selection and selection and the process is repeated as described in the art for identification by transgenic markers such as gus. (McCabe, et al. (1988) mo/Technology 6. 922-926). Shoots are induced in transgenic explants in hormone free medium The shoots are excised and roots are readily induced in similar growth medium. This is contrary to reports that shoots and roots are induced in media containing growth regulators. If some shoots have difficulty initiating roots after 2 to 3 weeks of separating from the explant, these shoots are placed in growth media containing 0.1 mg/l Indol butaric acid (IBA). Whole, mature, reproductive plants are thereby produced after transfer to greenhouse culture in soil.

The transformation and regeneration process of the present invention may be summarized as including the steps of selecting mature seeds, using explants of embryonic axes (excluding cotyledonary nodes) of Jack, Chapman, Williams 82, Pioneer 9341, Fayette, Peking, Corsoy and Biloxi cultivars, although it is emphasized herein that this process has proven itself to be and is intended to be disclosed as cultivar independent, . The seeds are sterilized with a halogen, preferably a gaseous or aqueous source of halogen, such as an aqueous source of chlorine The preferred sterilization method is chlorine gas. The seeds are soaked in a high BAP (50 mg/l or 100 mg/l 6-Benzylaminopurine) solution containing MS[(Murashige and Skoog 1962) media macro and micro salts supplemented with 1×B5 vitamins (Gamborg 1968)], for 3 hrs. The complete citations for these disclosures is Murashige T. and Skoog S. K. (1962) A revised medium for rapid growth and bioassays with tobacco tissue culture. Physiol. Plant. 15: 473-496; and Gamborg O. L. (1968), Miller R. A. and Ojima K. 1968. Nutrient requirement of soybean root cells. Exp. Cell. Res. 50: 151-158. The embryonic axes are isolated from the soaked seeds. Prepared embryonic axes tissues are incubated for 30 min. in a Agrobacterium solution for co-cultivation. Embryonic axes are then transferred to a filter paper wet with 100 mg/ml acetosyringone solution and placed in a petriplate. The petri plate with tissues are incubated at room temperature in the dark [no visible light, especially with no ultraviolet radiation] for 2 days. Tissues then are placed in semi solid (using a gelling agent) growth media containing MS (Murashige and Skoog 1962) macro and micro salts supplemented with 1×B5 vitamins (Gamborg 1968), antibacterial reagent to remove excessive Agrobacterium and relevant selection reagent to eliminate the none transformed tissue growth. The plates are maintained under 16/8 day light cycle. Every two weeks the tissues are transferred to fresh media After about 6 weeks all the explants with multiple shoots are transferred to MS media [MS (Murashige and Skoog 1962) macro and micro salts supplemented with 1×B5 vitamins (Gamborg 1968) and antibacterial reagent to remove excessive Agrobacterium and no selection reagent] in small culture tubes (100×13 mm Fisher®. The shoots grow out of the embryonic axes are separated and placed In the small culture tubes for root Initiation. Most of the separated shoots are rooted on MS media. The shoots took longer than 2 weeks to initiate roots are transferred to IBA media [MS (Murashige and Skoog 1962) macro and micro salts supplemented with 1×B5 vitamins (Gamborg 1968), 0.1 mg/l IBA and antibacterial reagent to remove excessive Agrobacterium and no relevent selection reagent]. The explants with multiple shoots are transferred to fresh MS media in tubes. This process carried out till all the shoots are harvested from the embryonic axes tissues.

The transformation and regeneration process of the present invention may be summarized as including the steps of selecting seeds (either mature or immature seeds), using explants of embryonic axes of cultivars. The seeds are sterilized with an aqueous source of sterilant (chlorine). The seeds are soaked in an aqueous solution or sterile water until germination of the seed. Placing the germinated seed on a wet paper support in a reduced light environment to continue germination. Providing agricultural growth media. Providing inoculation media at a pH of from 4.5-6.7 at a cell concentration/ml of 1.5×10⁷-1.5×109 for 10-200 minutes. Incubating the cells at 15-40° C. with an incubation medium on a wet porous (paper) support for 0.5 to 4 days. Washing after incubation is optional, with selection of active seeds made within seven days of completion of incubation. The selected seeds are contacted with a regeneration medium. The regenerating seeds are combined with a gelling agent. The regenerated seeds are then combined in an intermediate media with a gelling agent.

Mature seeds are surface sterilized with chlorine gas and soaked in a sterile growth regulator containing solution (to give a chemical treatment to the seeds) for 3 hrs. Harvest the embryonic axes (the tissue necessary) and insert the DNA [the method used was Agrobacterium]. Incubate the tissues in dark at room temperature on wet filter paper for 2 days.

### EXAMPLES

Protocol for Soybean Transformation

a) Mature seeds of soybean are placed on 100×20 perti plates in a single layer are arranged in a bell jar placed in the fume hood in such a way that all the plates are open and lids were set next to the plate and that there will be enough space for a small beaker holds little more than 100 ml of liquid

b) In a beaker that fits to the above description 100 ml of Clorox® is poured and placed in the bell jar. With the fume hood on and the bell jar lid ready to close, 3.5 ml of conc. hydrochloric acid was added to the beaker. Lid was closed immediately.

c) After overnight exposure to Cl⁻ gas the lid of the jar was open; lids of the petri plates were tipped on to close the petri plates without touching the interior of the petri plate or the lid. Closed petri plates were brought in to the laminar floor hood and left open for about 30 minuets to remove the excessive Cl⁻ gas build up.

Agrobacterium Culture Initiation

b) Inoculate LB [Luria Broth] liquid medium containing the appropriate{each antibiotic has a certain concentration that the bacteria that we are interested in can grow but kill the others}.amount of antibiotic with a scoop of Agrobacterium culture and grow overnight in an orbital shaker (250 rpm) at 28° C.

c) Next morning, 2 ml of the overnight grown Agrobacterium culture (in about 16 hr. Agrobacterium culture under these conditions grows to a concentration approximately 1.5×10⁸ cell per ml) to 23 ml of MS medium [Murashagi and Skoog 1962)] and then 100 micromoles of acetosyringon is added.

d) The culture is then placed in a 28° C. incubator shaker for 3 to 5 hours before being used for inoculation of explants.

a) Explant preperation and co-cultivation Dry sterilized seeds were soaked in 50 to 100 mg/l BAP (6-benzylaminopurine) containing MS (Murashige and Skoog 1962) liquid media for about 3 hours.

b) Soaked seed was positioned on a empty petri plate in such a way the embryo side up and away from your holding hand. Carefully look for the embryo attachment point to the cotyledons and make a deep insertion with a sharp #11 scalpel blade. Turn the scalpel blade over and push back the seed coat with the back of he blade to remove the embryonic axes with minimum damage. Place the explants In a petri-plate with the same liquid medium.

c) When all the explants are ready the water in the petri plate is replaced with Agrobacterium broth that is removed from the shaker just before inoculation.

d) Explants were allowed to soak for 30 minutes on the laminar floor hood.

e) After the 30 minuets Agrobacterium broth was drained off and explants were removed with a blunt object and placed on pre prepared incubation plates (Incubation plates were prepared by placing 2 layers of 3MM sterile filter paper on the bottom of the 100×20 petri plate and wetting the filter papers to the saturation with 100 μM Acetosyringone solution (in water).)

f) Plates with the explants are placed in a dark place at 22° C. to 24° C.+1.

Two days after inoculation, the explants are removed from the filter paper and placed on MS [MS (Murashige and Skoog 1962) macro and micro salts supplied with 1×B5 vitamins (Gamborg 1968), gelled with a gelling agent. In order to control the excessive Agrobacterium growth In the medium, 100 mg/ cefotaxim was added to the medium. In order to select the tissues that have the selection gene Inserted, relevant selection agent (depending on the selectable gene Inserted using the Agrobacterium) is included in the regeneration medium.

The following non-limiting examples are provided to assist in an appreciation of the invention and are not intended to limit the practice of terms or the scope of the present invention.

### EXAMPLE 1

Soybean seeds of cultivars Jack, Williams 82, Pioneer 9341, Fayette, Chapman Biloxi, Peking, corsoy were dry surface sterilized and soaked in 50 mg/l BAP media (MS (Murashige and Skoog 1962) macro and micro salts supplemented with 1×B5 vitamins (Gamborg 1968), 50 mg/l BAP) for 3 hours. Embryonic axes explants are isolated and co-cultured following the above explained procedure. After the two day incubation explants were placed on MSK (MS (Murashige and Skoog 1962) macro and micro salts supplemented with 1×B5 vitamins (Gamborg 1968), 100 mg/l cefotaxim and 50 mg/l kanamycin). During the first 2 weeks about 30% from the Initial explants were discarded due to selection. Rest of the explants were transferred to fresh MSK media. During the second transfer, (MSK media) all the explants that show any sign of shoot production (about 50% of the initial explants) were placed in new media. After about 6 weeks all the explants with multiple shoots (about 15% of the Initial explants) were transferred individually, to MS media (MS (Murashige and Skoog 1962) macro and micro salts supplemented with 1×B5 vitamins (Gamborg 1968) and 100 mg/l cefotaxim) in to small tubes.

Within few days in the small tubes, some explants elongated shoots that can be separated from the explant. Most of the separated shoots were rooted on MS media. The shoots taking longer than 2 weeks to initiate roots were transferred to IBA media (MS (Murashige and Skoog 1962) macro and micro salts supplemented with 1×B5 vitamins (Gamborg 1968), 0.1 mg/l IBA and 100 mg/l cefotaxim). The explants with multiple shoots were transferred back to MS media in tubes. When the large shoots were removed the remaining shoots elongate.

Leaves from some shoots were harvested for PCR directly from the tubes at the time they were separated from the explant. First set of plants were transplanted in Jiffy pots (Universal Mix® Strong-Lite Horticultural Products). About 10 weeks after explant initiation. This process was continued over few months. Only the PCR positive plants were maintained in the greenhouse to the maturity. Matured plants were harvested and random samples from selected plants were germinated to analyze for PCR and Southerns.

What is claimed:

1. A method for transforming soybean comprising:
a) inserting a functional gene into a cell of a soybean explant of a soybean seed;
b) explants of the soybean seeds developed from step a) are then transferred to a porous substrate for incubation;
c) after incubation, the explants are transferred to a media of macro and micro salts supplemented with 1×B5 vitamins; and
d) after shoot elongation, separated shoots, with or without roots, are either
i. transferred to soil; or
ii. contacted with at least 1 mg/l IBA before transplant.

2. The method of claim 1 wherein a soybean is first pretreated with hormone before step a).

3. The method of claim 2 wherein the hormone is a cytokinin preferably 6-benzyleaminopurine.

4. The method of claim 2 wherein the hormone comprises at least about 40 mg/L of 6-benzyleadinopurine for at least 1.5 hours.

5. The method of claim 1 wherein Agrobacterium is used for inserting the functional gene into the cell.

6. The method of claim 5 wherein embryonic axes explants of the soybean seeds are developed in claim 1 step a) and are transferred to the porous substrate.

7. The method of claim 6 wherein the transfer to the porous substrate is carried out in the presence of at least one phenol compound naturally produced when plant cells have been wounded, to induce vir genes.

8. The method of claim 7 wherein incubation is in the dark in the presence of the at least one phenol compound at 20° C.-25° C. for at least 24 hours.

9. The method of claim 1 step c) wherein the shoot regeneration is performed in the presence of an antibiotic to control Agrobacterium growth.

10. The method of claim 9 wherein the shoot regeneration is performed in the presence of a selection agent.

11. The method of claim 10 wherein the selection agent comprises an antibiotic or a herbicide or a growth inhibitor.

12. The method of claim 11 wherein the selection agent is chosen from the group consisting of kanamycin, bialophos, hygromycin, and glyphosate.

13. The method of claim 1 wherein the porous medium contains a signal compound.

14. The method of claim 5 wherein the porous medium contains a signal compound.

15. The method of claim 7 wherein the porous medium contains a signal compound.

16. The process of claim 14 wherein the signal compound is selected from the group consisting of acetosyringone, alpha-hydroxyacetosyringone, acetovillone, syringaldehyde, syringic acid, sinapinic acid, and mixtures thereof.

17. The process of claim 1 wherein the porous medium comprises paper.

18. The process of claim 5 wherein the porous medium comprises paper.

19. A method of pretreating soybean tissue for development into a transgenic soybean plant comprising:
a) Pre-treating sterile seeds with a relatively high concentration of at least about 40 mg/L of 6-benzyleaminopurine for at least 1.5 hours.

20. The method of claim 19 wherein the relatively high concentration of a solution of 6-bezyleaminopurine between about 40-100 mg/L.

21. The method of claim 20 wherein the pretreatment is performed for at least 2.5 hours.

22. The method of claim 20 wherein after the pretreatment, an explant derived from embryonic axes of a mature soybean seed is co-cultivated with Agrobacterium species containing a chimeric gene in the presence of liquid plant tissue culture media.

23. The method of claim 20 wherein after the pretreatment, an explant derived from embryonic axes of a mature soybean seed is co-cultivated with Agrobacterium species containing a chimeric gene in the presence of plant media.

24. The method of claim 22 wherein virulence is induced in the Agrobacterium by incubating the tissues at a temperature between 20° C.-25° C.

25. The method of claim 22 wherein virulence is induced in the Agrobacterium by incubating the tissues at a temperature between 22° C.-24° C.

26. The process of claim 22 wherein co-cultivation is performed on porous support media.

27. The process of claim 26 wherein the porous support medium comprises paper.

28. The process of claim 26 wherein the porous support medium contains a signal compound.

29. The process of claim 28 wherein the porous support medium contains a signal compound selected from the group consisting of acetosyringone, alpha-hydroxyacetosyringone, acetovanillone. syringaldehyde, syringic acid, sinapinic acid, and mixtures thereof.

30. The process of claim 2 wherein the pretreatment is effected with an effective amount of a cytokinin.

31. The process of claim 30 wherein the cytokinin is present in a concentration of at least 20 mg/L.

32. The method of claim 1 wherein the insertion of the functional gene into the cell is done by a mechanical or physical DNA delivery method.

33. The method of claim 32 wherein the mechanical or physical DNA delivery method is selected from the group consisting of biolistic delivery, whisker delivery, or electroporation.
